(19) United States
(12) Patent Application Publication
(10) Pub. No.: US 2025/0281315 A1
(43) Pub. Date: Sep. 11, 2025

(54) AN INTERFACE FOR A WEARABLE ASSISTANCE DEVICE

(71) Applicant: Vanderbilt University, Nashville, TN (US)

(72) Inventors: Karl E. Zelik, Nashville, TN (US); Jaime S. Fine, Nashville, TN (US)

(21) Appl. No.: 18/859,836

(22) PCT Filed: Apr. 26, 2023
(86) PCT No.: PCT/US2023/066231

§ 371 Date: Oct. 24, 2024

Related U.S. Application Data
(60) Provisional application No. 63/335,109, filed on Apr. 26, 2022.

(51) Int. Cl. A61F 5/02

(52) U.S. Cl. CPC A61F 5/02

(57) ABSTRACT

An interface for a wearable assistance device that comprises a sleeve that comprises a main body configured to at least partly surround a user's body segment and is adjustable for comfort and fit. The interface has an elastic portion. A stop is positioned adjacent to the elastic portion such that the stop bridges the elastic portion. The stop is configured to restrict an amount of stretch that the elastic portion affords the sleeve beyond a set length. An engagement feature configured to connect the interface to a component of the wearable assistance device.

### CROSS REFERENCE TO RELATED APPLICATION

This application claims the benefit of priority of U.S. Provisional Application Ser. No. 63/335,109 filed on Apr. 26, 2022, and entitled “Exosuit Body Interface Having a Multi-Point Anchor System, Hard Stop, and/or Magnetic Fastener,” the content of which is relied upon and incorporated herein by reference in its entirety.

### GOVERNMENT SPONSORSHIP

This invention was made with government support under grant W911NF2120078 awarded by the U.S. Army. The government has certain rights in the invention.

### FIELD OF THE INVENTION

The present disclosure is in the field of wearable assistance devices, such as exosuits/exoskeletons. More particularly, the present disclosure relates to wearable assistance devices for reducing muscle stress, fatigue, injury and pain in the lower back or other body segments or parts.

### BACKGROUND

Exoskeletons and exosuits are defined as wearable devices that augment, enable, assist, or enhance motion, posture, or physical activity. Exosuits biomechanically assist tasks ranging from bending, lifting, and reaching to walking, running, and jumping. Exosuits are used in a range of different applications, such as clinical and occupational. Exosuit research has primarily focused on device design and validation that exosuits reduce muscular demands, effort, or fatigue. Despite the focus on exosuits as physical assistance devices, the key barriers to societal adoption are frequently related to comfort, fit, and freedom of movement. Recent studies have begun to assess physical comfort thresholds when exosuit forces are applied to different parts of the body, identifying that comfort limits increase with habituation and padded materials. Nevertheless, designing comfortable body interfaces and achieving physical comfort when assistive forces are applied is deceptively difficult, and remains a major issue for many exoskeletons and exosuits. In addition, static, dynamic, and cognitive fit characteristics have been defined for consideration during exosuit development and evaluation.

However, one area that has received little attention from the exosuit research community is thermal comfort, which is defined as a person's satisfaction with their thermal environment. Thermal comfort is influenced by thermodynamics (the physics of heat and temperature) and thermoregulation (the process by which the body maintains its temperature). While the issue of thermal comfort with exosuit use is somewhat known, the most compelling motivation comes directly from exosuit early adopters in industry who have commonly reported thermal comfort as a barrier to user adoption.

Maintaining thermal comfort is challenging since exosuits must attach to the body to apply assistive forces, and these physical body interfaces (termed interfaces) inevitably trap some amount of heat. Heat retention is due, in part, to the lack of space between the skin and interface which inhibits airflow, the transfer of heat generated by the body, and evaporation of sweat to the surrounding environment. Similar to other wearable accessories (e.g., backpacks, performance apparel), thermal comfort can be partially improved through material selection and design. For instance, minimizing the size of the interface can reduce the surface area where heat is retained. However, if an interface (e.g., sleeve, cuff) is too small or thin, it can become physically uncomfortable under load due to pressure points on the skin, or too structurally weak to transmit exosuit forces to the body. Consequently, exosuit interface design aims to balance thermal and physical comfort with adequate material strength to apply requisite forces.

Thus, it is desirable to provide an exosuit interface and method of using an exosuit interface that are able to overcome the above-described disadvantages. For example, a leg/thigh sleeve such as the type disclosed in International Publication WO 2021/257671 may incorporate and benefit from any one of the Multi-Point Anchor System, Hard Stop, and Magnetic Fastener, and other devices, systems, and methods, described in this disclosure.

Advantages of the present disclosure will become more fully apparent from the detailed description of the invention hereinbelow.

### SUMMARY

Accordingly, the present disclosure may provide an interface for a wearable assistance device that comprises a sleeve that comprises a main body configured to at least partly surround a user's body segment, the main body having at least one elastic portion, a stop positioned adjacent to the at least one elastic portion such that the stop bridges the at least one elastic portion, wherein the stop is configured to restrict an amount of stretch that the elastic webbing affords the sleeve beyond a set length, and an engagement feature configured to engage a component of the wearable assistance device.

In certain examples, the stop is an inelastic webbing; the stop is an elastic material having a stiffness greater than a stiffness of the at least one elastic portion; a length of the stop is longer than a length of the at least one elastic portion such that the stop has slack when not in use; the main body includes a top, a bottom, and opposite ends, and a length defined between the opposite ends, the length being longer than a height of the sleeve defined between the top and bottom; and/or the stop is located at the bottom of the main body;

In other embodiments, the opposite sleeve ends are configured to connect to one another; a middle portion of the main body is wider than the first and second ends, and the engagement feature is located at the middle portion and extends away from the bottom of the main body; the engagement feature is a release buckle or a carabiner attached to the sleeve; the interior side of the main body is formed of non-slip fabric; the main body includes at least one ventilated panel; and/or the sleeve is a thigh sleeve.

The present disclosure may also provide an interface for a wearable assistance device, that comprises a sleeve that comprises a main body configured to at least partially surround a user's body segment, and the main body has at least one elastic portion, at least one adjustment feature is coupled to the main body, and the adjustment feature comprises a webbing or strap with a free end portion thereof, and a strap management feature is positioned on the main body. The strap management feature is configured to removably secure the free end portion to the sleeve. The main body also has an engagement feature that is configured to engage a component of the wearable assistance device. The free end portion is configured to tighten the sleeve on the user's body segment upon securing the free end portion via the strap management feature. The adjustment feature is configured to limit an amount of loosening of the sleeve.

In an example, the free end portion of the strap can be a pull tab. In an example, the strap or webbing runs through a strap adjuster wherein actuation of the strap adjuster removes the pull tab from the strap management feature to thereby loosen the sleeve on the user's body segment and the loosening of the sleeve is limited by the pull tab contacting the strap adjuster. In another example, a stop is positioned adjacent to the at least one elastic portion such that the stop bridges the at least one elastic portion, wherein the stop is configured to restrict an amount of stretch that the elastic webbing affords the sleeve beyond a set length.

In some examples, the strap management feature is configured to removably secure the pull tab to an exterior side of the main body; the strap management feature is a strap segment spanning a width of an end of the main body; the strap management feature removably secures the pull tab via magnetic attraction between a magnet positioned in or on the pull tab and a metal plate in or on the main body, or vice versa; and/or the metal plate is a first metal plate, wherein the interface further comprises a second metal plate also positioned in or on the main body at a location that is different than the position of the first metal plate in or on the main body, wherein the pull tab is configured to be removably secured to the main body via magnetic attraction between the magnet and the first metal plate, when tightening the sleeve around the body segment, and wherein the pull tab is configured to be removably secured to the main body via magnetic attraction between the magnet and the second metal plate, when loosening the sleeve around the body segment.

In other examples, one or more anchors are configured to connect or suspend the sleeve from the user's clothing, belt, or other body-worn gear; one of the anchors is positioned along an interior side of the sleeve and another one of the anchors is positioned along an exterior side of the sleeve; the strap or webbing of the adjustment feature is provided at one end of the sleeve; and/or an adjustable strap is provided at the other end of the sleeve.

This summary is not intended to identify essential features of the claimed subject matter, nor is it intended for use in determining the scope of the claimed subject matter. It is to be understood that both the foregoing general description and the following detailed description are exemplary and are intended to provide an overview or framework to understand the nature and character of the disclosure.

### BRIEF DESCRIPTION OF THE FIGURES

The accompanying drawings are incorporated in and constitute a part of this specification. It is to be understood that the drawings illustrate only some examples of the disclosure and other examples or combinations of various examples that are not specifically illustrated in the figures may still fall within the scope of this disclosure. Examples will now be described with additional detail through the use of the drawings, in which:

FIG. 1 is a plan view of an interface according to a first embodiment of the present disclosure, showing an exterior side of the interface;

FIG. 2A is a partial enlarged view of the interface illustrated in FIG. 1, showing a stop incorporated into the interface;

FIG. 2B is a view of an alternative stop for the interface illustrated in FIG. 1;

FIGS. 3A and 3B are images of the interface illustrated in FIG. 1, worn by a user and connected to a wearable assistance device also worn by the user, showing the user in standing and squatting positions, respectively.

FIGS. 4A and 4B are partial enlarged views of the interface illustrated in FIG. 1, showing an adjustment and strap management features at an end of the interface;

FIG. 5 is a partial enlarged view of the interface illustrated in FIG. 1, showing an engagement feature for connecting to a wearable assistance device

FIGS. 6A and 6B are images of the interface illustrated in FIG. 1, showing the interface as thigh sleeves worn by the user in tightened and loosened configurations, respectively;

FIGS. 7A and 7B are partial enlarged end views of the interface illustrated in FIG. 1, showing the tightened and loosened configurations, respectively;

FIG. 8 is a partial enlarged end view of the interface illustrated in FIG. 1, showing a strap management feature at one end of the interface;

FIG. 9 is a partial enlarged end view of the interface illustrated in FIG. 8, showing an adjustment feature at the other end of the interface;

FIGS. 10A and 10B are plan views of an interface according to a second embodiment of the present disclosure, showing the exterior side of the interface;

FIG. 10C is a partial enlarged end view of the interface illustrated in FIGS. 10A and 10B, showing an adjustment feature at one end of the interface;

FIG. 10D is a partial enlarged end view of the interface illustrated in FIGS. 10A and 10B, showing the other end of the interface opposite to that shown in FIG. 10C;

FIGS. 11A and 11B are plan views of the interior side of the interface illustrated in FIGS. 10A and 10B

FIG. 11C is a partial end view of the interface illustrated in FIGS. 11A and 11B; and

FIGS. 12A and 12B are images of the interface illustrated in FIGS. 10A and 10B, worn by a user and connected to a wearable assistance device also worn by the user, showing the user in standing and squatting positions, respectively.

### DETAILED DESCRIPTION

It is to be understood that the figures and descriptions of the present disclosure may have been simplified to illustrate elements that are relevant for a clear understanding of the present disclosure, while eliminating, for purposes of clarity, other elements found in a typical wearable assistance device or typical method of using a wearable assistance device. Those of ordinary skill in the art will recognize that other elements may be desirable and/or required in order to implement the present disclosure. However, because such elements are well known in the art, and because they do not facilitate a better understanding of the present disclosure, a discussion of such elements is not provided herein. It is also to be understood that the drawings included herewith only provide diagrammatic representations of the presently preferred structures of the present disclosure and that structures falling within the scope of the present disclosure may include structures different than those shown in the drawings. Reference will now be made to the drawings wherein like structures are provided with like reference designations.

It should be understood that the inventive concepts set forth herein are not limited in their application to the construction details or component arrangements set forth in the following description or illustrated in the drawings. It should also be understood that the phraseology and terminology employed herein are merely for descriptive purposes and should not be considered limiting.

It should further be understood that any one of the described features may be used separately or in combination with other features. Other invented devices, systems, methods, features, and advantages will be or become apparent to one with skill in the art upon examining the drawings and the detailed description herein. It is intended that all such additional devices, systems, methods, features, and advantages be protected by the accompanying claims.

For purposes of this disclosure, the phrase “body segment” may include a body part such as a back, lumbar spine, hip, neck, etc., or a body joint such as an ankle, knee, elbow, wrist, etc., and thus, may all be used interchangeably. Also, the phrase “body segment” may include multiple body parts or body joints.

For purposes of this disclosure, the phrase “mechanical advantage” may include “assistive force”, “assistive torque”, or “assistive moment of force”, and thus, may all be used interchangeably.

For purposes of this disclosure, the phrase “wearable assistance device” may be an exosuit, exoskeleton, prosthesis, orthosis, or other device that provides mechanical advantage about a body segment of a user, and may be passive, quasi-passive, or powered. The phrase is used broadly, and also may include rehabilitative or resistive devices that are intended to assist, augment, support and/or train users through mechanical interaction forces exerted by the device.

For purposes of this disclosure, the phrases “elastic band”, “elastic element”, and “elastic member” may be used interchangeably, and may be any member that has an amount of elasticity associated with it and which can take the form of, for example, a spring, cable, string, strap, cord, webbing, rope, band, beam, gas-spring, pneumatic, carbon fiber, fiber glass, etc., and may be coiled or non-coiled. The elastic element may have a linear or non-linear stiffness profile.

For purposes of this disclosure, the phrases “upper body interface” and “lower body interface” refer to body interfaces that can be positioned anywhere on the user's body, with the upper body interface placed higher relative to the lower body interface, assuming the user is in a standing/vertical position. The term “body interface” or “interface” refers to the component or components of a wearable assistance device that physically connect or attach to a body segment of a user. An interface may consist of, for instance, a sleeve (of, for example, leg/thigh type), cuff, vest, shirt or pants.

For purposes of this disclosure, the phrase “clutch mechanism” or “clutch” may include any device that engages and disengages mechanical elements (e.g., elastic members or portions thereof) that bear or transmit force or mechanical power. A clutch mechanism may be unpowered such that it engages and disengages based on manual input or movement from the user and may include one or more springs that bias the clutch towards a nominal mode (e.g., on or off). Alternatively, a clutch mechanism may be powered such that a motor or other actuator with its own power supply is used to control engagement and disengagement. Additionally, a motor or other actuator may be used to control the position of clutch engagement relative to one or more mechanical elements (e.g., elastic members), or to control the set point of an elastic member relative to the position of clutch engagement, thereby adjusting or setting tension of, for example, elastic member(s). The clutch mechanism may be used in combination with additional motors or other actuators that provide tensile force or perform work along (parallel with) the elastic members or provide a force transverse or perpendicular to the elastic members. The engaging and disengaging by the clutch mechanism of a mechanical element may be achieved by any form of clutch or brake or mechanism providing a similar function, for example, a ratchet, dog clutch, cam clutch, friction clutch, overrunning clutch, disc brake, drum brake, latch, buckle, variable-ratio gear, or other resecurable fastening device.

For purposes of this disclosure, the phrase “leaning” is interchangeable with the terms “hinging”, “flexing”, “extending”, “bending”, “stooping”, and/or any combination of those movements or postures.

For purposes of this disclosure, the phrase “cable” is interchangeable with the terms “string”, “strap”, “cord/cording”, “webbing”, “rope”, “band”, and/or any combination thereof.

This disclosure describes an improvement for interfacing wearable assistance devices to the body, with some examples focused on the thighs. Examples of the interface described in this disclosure ensure exosuit physical comfort, thermal comfort, and effectiveness (ability to provide force assistance). The interface described in examples in this disclosure is for a back-assist exosuit, but this same or similar interface could also be applied to other types of exoskeletons/exosuits and on other body parts.

Referring to the figures, the present disclosure teaches interfaces 100, 100′ for a wearable assistance device that is designed for proper fit, freedom-of-movement, and comfort, including thermal comfort for the user. In general, the interface may comprise a sleeve that is configured to at least partially surround a user's body segment, such as the thigh, and has an engagement feature configured to engage a component of the wearable assistance device. The sleeve may comprise at least one elastic portion and a stop that bridges the elastic portion. The stop is configured to restrict the stretch of the elastic portion and consequently the sleeve beyond a set length in order to maintain the assist force provided by the wearable assistance device. The interface may also include adjustment features for allowing easy loosening and tightening of the sleeve for the proper fit and optimal comfort for the wearer. This tightening mechanism would be used when switching from unassisted or disengaged mode, to put the wearable assistance device in a state ready for assistive force to be provided.

The design features of the interface of the present disclosure, either separately or in combination, provide physical comfort, thermal comfort and assistive effectiveness in interfacing exosuits with the body, while mitigating snag risks or movement interference. Some of those design features may include a quick and easy-to-use loosening mechanism for the body interface to enhance thermal comfort; and/or a quick and easy-to-use tightening mechanism for the body interface to configure and secure an exosuit to provide assistance, with an integrated strap management solution to avoid snag risks. Another design feature of the interface may be a multi-point anchor system. In one embodiment, this multi-point anchor system includes two thigh sleeve-to-waist-belt anchors per leg located on the inner and outer thigh to ensure physical comfort and prevent movement interference by balancing/stabilizing the thigh sleeve when in a loosened state and preventing the thigh sleeve from sliding/sagging down the leg. Yet another design feature of the interface is an elastic portion along with an integrated stop to achieve physical comfort when exosuit assistance is provided.

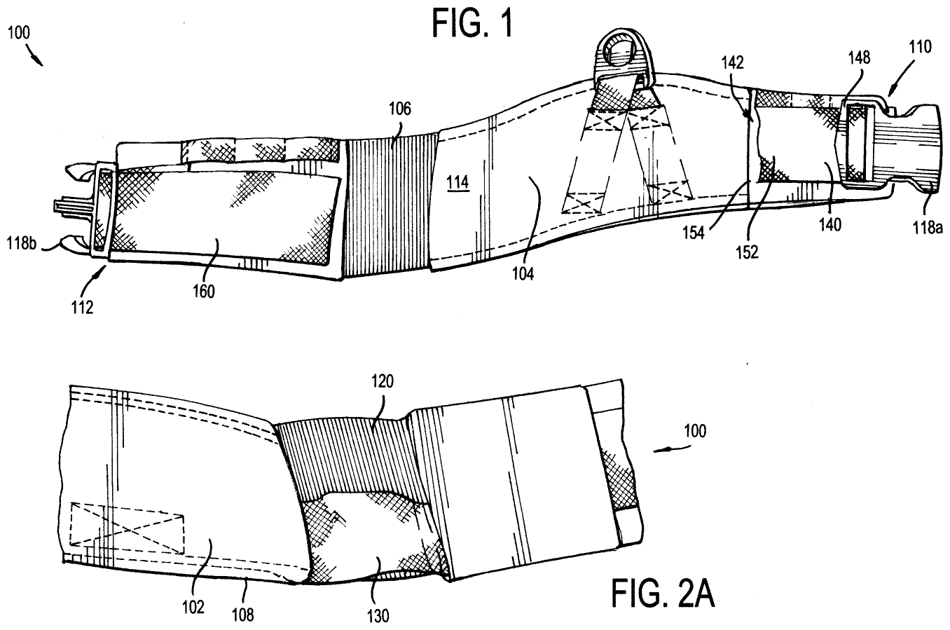

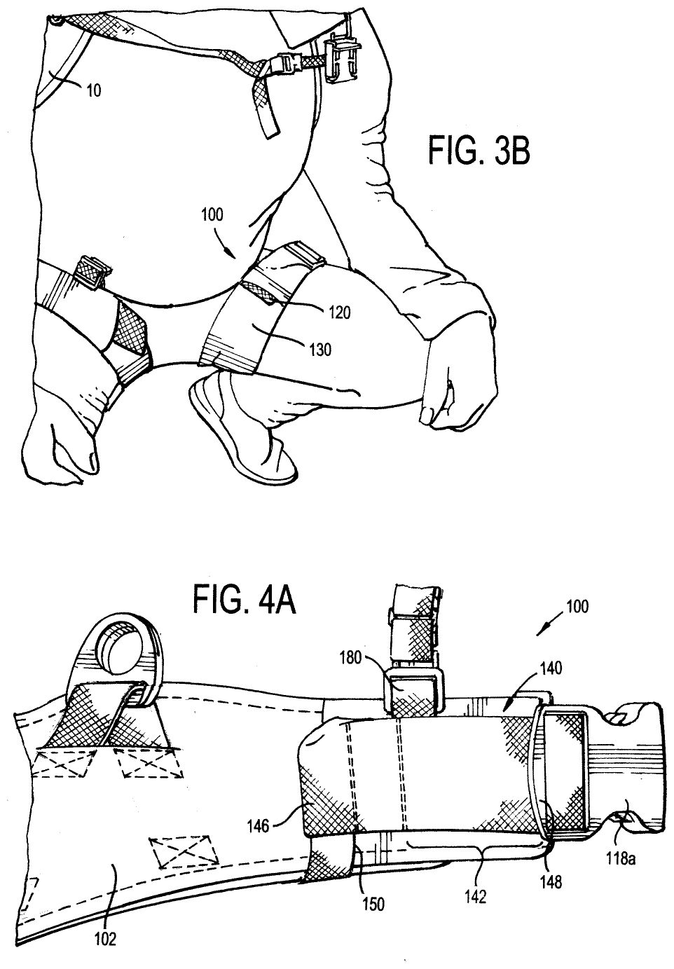

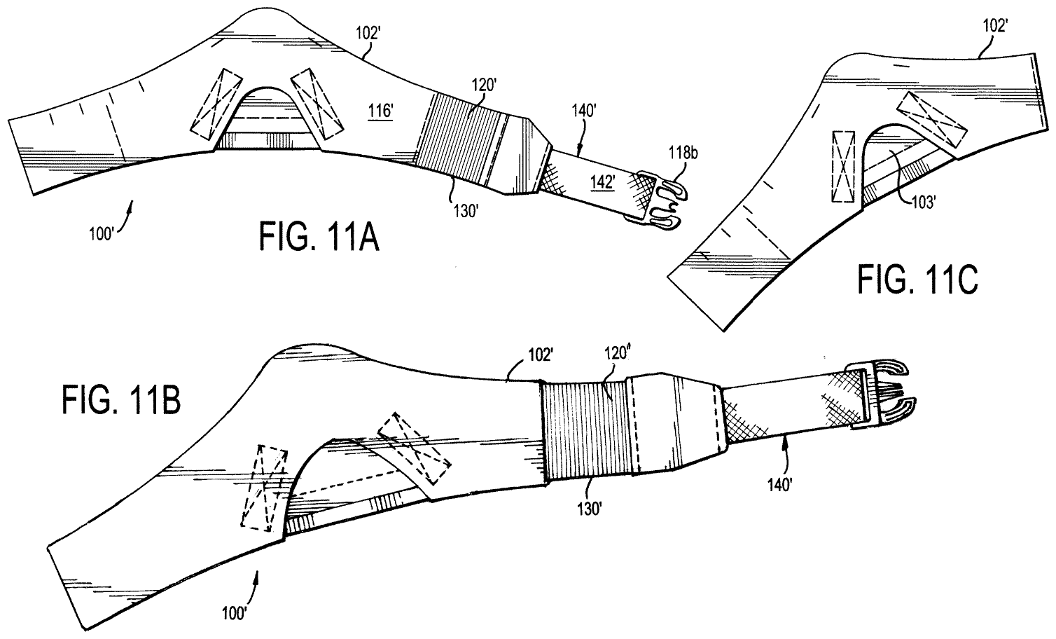

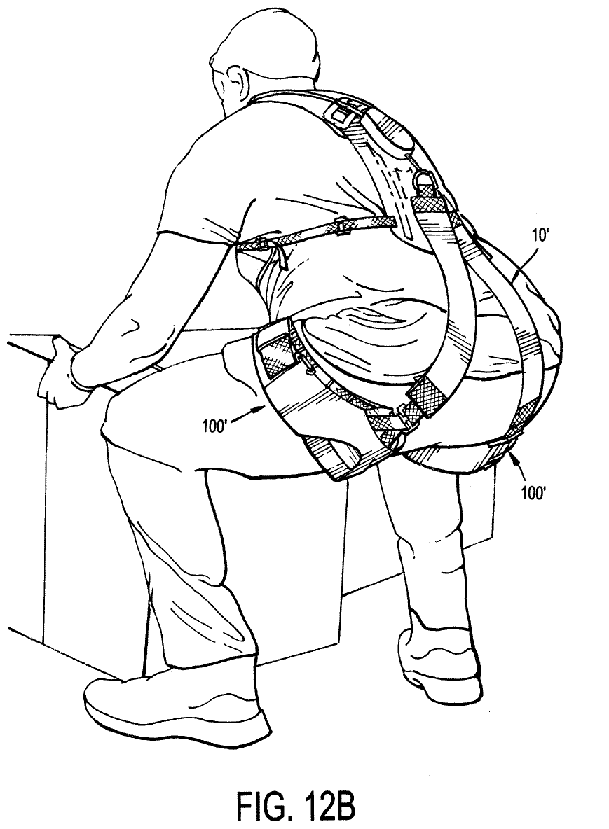

FIGS. 1 thru 9 illustrate an interface 100 of a first exemplary embodiment of the present disclosure. FIGS. 10A thru FIG. 12 illustrate an interface 100′ of a second exemplary embodiment. Interface 100 or 100′ may comprise a sleeve 102 or 102′ configured to be worn on a body segment and configured to be connectable to a wearable assistance device 10. In an example, one interface 100 or 100′ is worn on each thigh of the user and each interface 100 or 100′ is connected to a component, such as an elastic strap or band, of the assistance device 10 worn by the user, such as shown in FIGS. 3A, 3B, 6A, and 6B and FIGS. 12A and 12B.

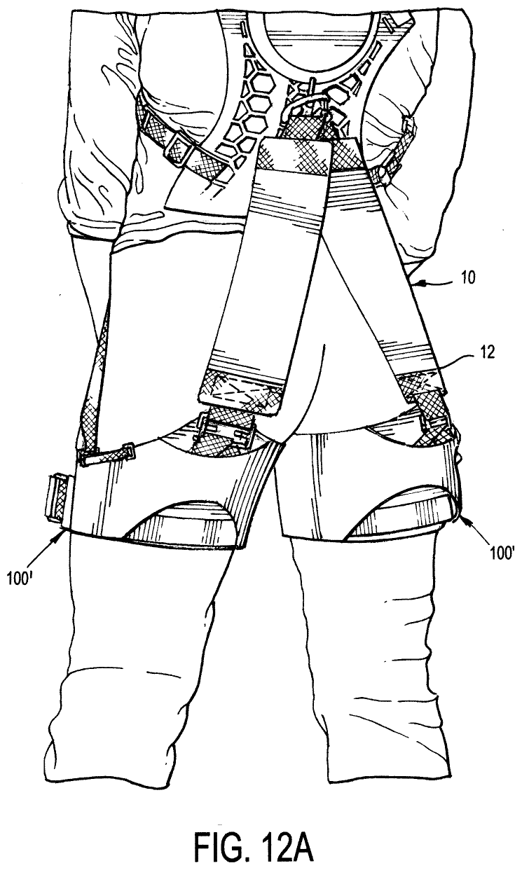

Sleeve 102 of the first embodiment may comprise a main body 104 that has a top 106, a bottom 108, opposite first and second ends 110 and 112, a length L defined between the first and second ends, an exterior side 114, and an interior side 116. The length L of the sleeve 102 may be elongated, that is the length L is longer than a height/width H of the sleeve 102. The main body 104 of the sleeve 102 may be one or more layers or panels of foam and/or fabric that are positioned over the back of the leg when worn as a thigh sleeve. The first and second ends 110 and 112 are configured to be connectable to and detachable from one another, such as via a buckle or snap engagement. A middle portion of the main body can be wider than the first and second ends 110 and 112. An adjustment feature 140, such as a strap or webbing, can be provided at one or both of the first and second ends 110 and 112 for sizing and fit adjustment for the user. Sleeve 102′ of the second embodiment is generally the same as the sleeve 102 of the first embodiment, except for some variations including the sleeve 102′ being taller/wider than the sleeve 102 and the sleeve 102′ routes forces in a taller triangle shape or configuration of the main body 104′ than the sleeve 102, as shown in FIGS. 10A and 11A. Both sleeves reduced motion on the user and improved comfort and force transfer.

The main body 104 or 104′ of the sleeve 102 or 102′ has an elastic portion 120 or 120′ that facilitates stretching of the interface 100 or 100′ when worn circumferentially around a body segment, such as around the thigh. The elastic portion 120 or 120′ provides for physical comfort of the user and may be an elastic fabric, elastic textile, elastomer or the like. The elastic portion may be one layer or multiple layers. The elastic portion may have a non-linear stiffness profile. The elastic portion may vary in stiffness along its width or height. For instance, in some embodiments, the elastic portion could be stiffer near the bottom and less stiff near the top.

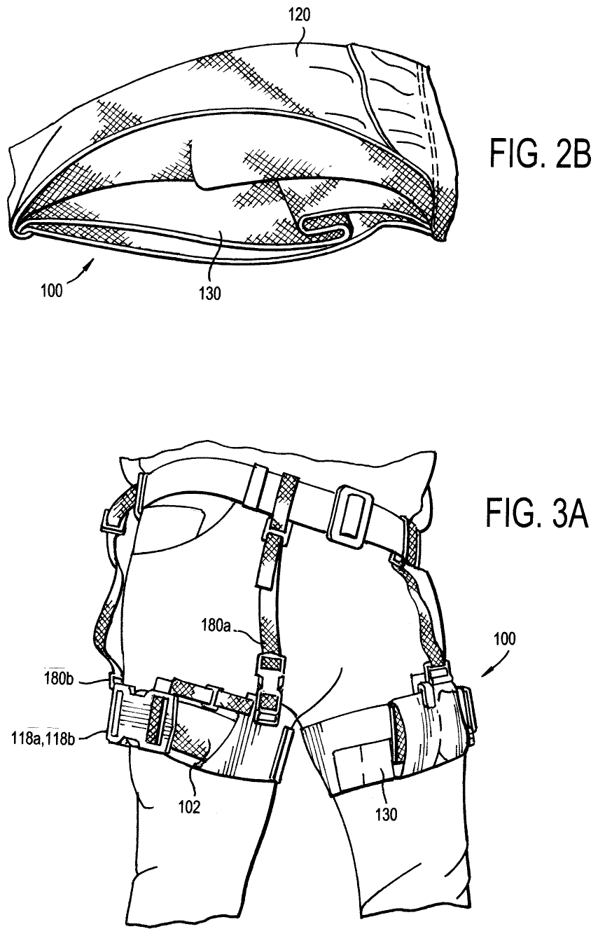

A stop 130 or 130′ is incorporated into the main body 104 or 104′ of the sleeve 102 or 102′ to reduce or prevent lengthening of the elastic portion 120 or 120′ of the sleeve 102 or 102′ beyond a set length. The length is set to accommodate muscle bulging and body segment shape changes during movement, for instance, which is often on the order of 0.5 to 1.25 inches for the thigh. The stop prevents overstretching of the interface 100 or 100′ which could result in negating the assist force being provided by the wearable assistance device. In an example, the stop 130 or 130′ can be an inelastic webbing located at the bottom 108 or 108′ of the sleeve 102 or 102′, as seen in FIG. 1 showing the stop 130 and FIGS. 10A and 11A showing the stop 130′. The stop 130 or 130′ can be located on either the exterior side 114 or 114′ of the sleeve 102 or 102′ and the interior side 116 or 116′ of the sleeve 102 or 102′. The stop 130 or 130′ can be configured to bridge the elastic portion 120 or 120′, as best shown in FIGS. 2A and FIGS. 10A and 10B, thereby limiting stretching of the elastic portion 120 or 120′ and the sleeve 102 or 102′ beyond the length of the stop 130 or 130′. In an example, the stop 130 of the sleeve 102 may be an inelastic webbing that has a length that is longer than a length of the elastic portion 120 such that the ends of the stop 130 define the bridge over the elastic portion 120. In another example, the stop 130′ of the sleeve 102′ may be elastic strap or webbing but with a higher stiffness than the elastic portion 120′. The stop could be the same length as the elastic portion, or shorter, or longer.

When the length of the stop 130 or 130′ is longer (in a direction parallel to the length L of the sleeve 102 or 102′) than the length of the elastic portion 120 or 120′, the stop or 130 or 130′ has a slack or slack length when bridging the elastic portion 120. When the interface 100 or 100′ is not stretched, such as when the user is standing and relaxed, as seen in FIGS. 3A and 12A, the stop 130 or 130′ has slack because the elastic portion 120 or 120′ is not stretched. When the sleeve 102 or 102′ is stretched (such as when the user squats as seen in FIG. 3B and FIG. 12B), the slack or slack length of the stop 130 or 130′ is eliminated, such that the stop 130 or 130′ is taut, thereby limiting the stretching capacity of the sleeve 102. In an example, the slack length may be around 0.5 to 1.25 inches for the thigh, but it may also be outside this range for the thigh or other body segments.

The stop 130′ of the interface 100′ which is elastic provides a softer stop than the stop 130. The elastic of the stop 130′ can have a higher elastic stiffness relative to the elastic webbing 120′. The length of the stop 130 or 130′ can be fixed or adjustable based on end-user or design needs. The stop of either embodiment can be either a hard stop, such as nylon webbing or a soft stop, such as elastic fabric, which both limit the lengthening of the sleeve, except with different stiffness levels. Or the stop can be designed into the elastic webbing 120 or 120′, for instance, by creating an elastic fabric panel with a stiffness profile that varies along its height or length.

In one example, the elastic portion 120 or 120′ of the sleeve 102 or 102′ can be located on the inner leg and part of a thigh sleeve for an exosuit. In another example, more than one elastic portion 120 or 120′ may be incorporated into the sleeve 102, such as one portion configured over the inside (medial) part of the user's leg and closer to one end 112, and one configured over the outside (lateral) part of the user's leg and closer to the other end 110. In another example, more than one elastic portion 120 or 120′ may be incorporated into the sleeve 102 or 102′, such as one on the exterior side 114 or 114′ and one on the interior side 116 or 116′ (that is against the body segment) of the sleeve 102 or 102′, where each elastic portion 120 or 120′ has its own stop 130 or 130′. In an example, the height/width of the stop 130 or 130′ is less than the height/width of the elastic portion 120 or 120′ of the sleeve 102 or 102′. For example, the height/width of the stop 130 or 130′ can be about half or less than half of the height/width of the elastic portion 120 or 120′. In an example, the height/width of the stop 130 or 130′ is about 1.5 inches and the height/width of the elastic portion 120 or 120′ is about 3 inches.

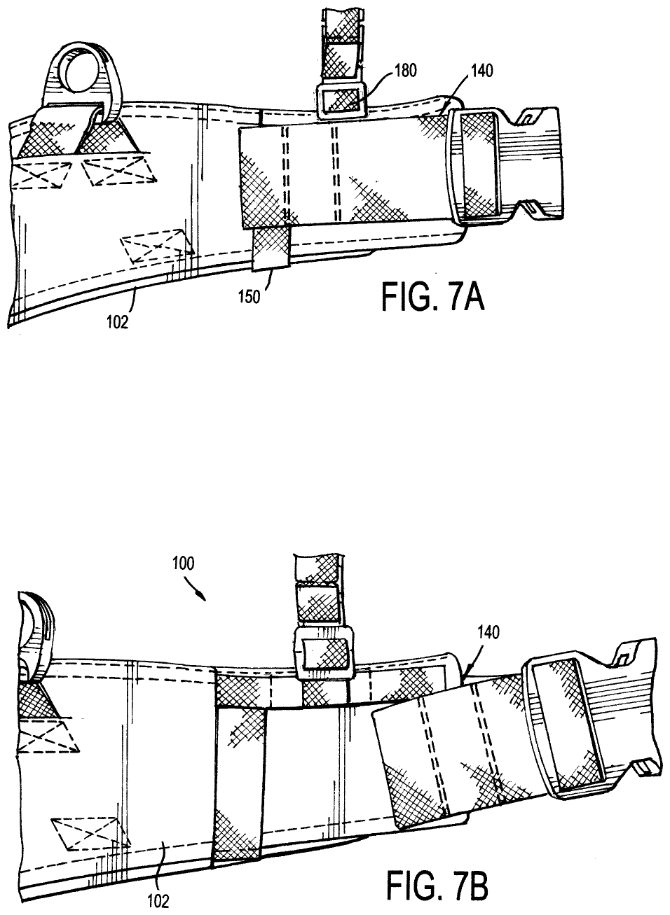

The elastic portion 120 or 120′ is configured to stretch in at least the circumferential direction around a body segment to provide comfort in the leg/thigh sleeve during squats and leans, as seen in FIG. 6B and FIG. 12B. During squats, leans, and other movements, the elastic portion 120 or 120′ of the sleeve 102 or 102′ stretches around the body segment, e.g. thigh, but the total amount of stretch is limited by the stop 130 or 130′. The sleeve's elastic portion 120 or 120′ also accommodates the changes in the size and shape of the leg during various movements (e.g., due to muscular bulging). The stop 130 or 130′ placed along at least the bottom 108 or 108′ of the sleeve 102 or 102′ prevents the sleeve 102 or 102′ from overstretching under load (e.g., when the wearable assistance device, such as an exosuit, is providing assistive force), and allows the sleeve 102 or 102′ to better conform to the tapered shape of the thigh, for example. That configuration may also provide a better fit across the gender spectrum.

The stop 130 or 130′, whether hard or soft, such as described herein, can be used in any interface 100, including a thigh interface, for an exosuit, and has the potential to be used in other exosuit interfaces that serve as an interface on other parts of the body (e.g., shank, arm, trunk). In other examples, the stop 130 of the sleeve 102 can be placed directly against the leg/thigh and/or on top of the elastic portion 120. In another example, the stop 130 can be sandwiched in the middle of multiple elastic layers (FIG. 2B). That is, the stop 130 is the middle layer between two elastic layers. That will hide the stop, thereby minimize the snag risk of the stop 130.

Multiple elastic layers may be used to achieve a desired or higher level of elastic stiffness than would be provided with a single layer of elastic (e.g., webbing). Other types of elastic/springs (e.g. bungee cord) and other types of materials for the stop 130 or 130′ can be used, depending on the application. The stop 130 or 130′ could be made from textiles (e.g., webbing) or from other materials (e.g., elastomers, metals). The stop 130 or 130′ can be located at the bottom of the sleeve when the sleeve is on the thigh; or the stop 130 or 130′ may be located in the middle and/or at the top of the sleeve, or even over the entire height/width H of the interface 100 or 100′, when the interface 100 or 100′ is applied to the thigh or other body segments.

The first and second ends 110 and 112 are configured to be connectable to one another, such as via a buckle, e.g. female and male buckle components 118a and 118b, a snap engagement, or the like, to attach the interface 100 to the body segment. In the example of the interface 100 being used as a thigh sleeve, the engagement of the buckle components 118a and 118b would be located on the outside of the thigh when worm by the user. Similar to the first embodiment, the ends of the sleeve 102′ connect to one another, such as via female and male buckle components 118a and 118b.

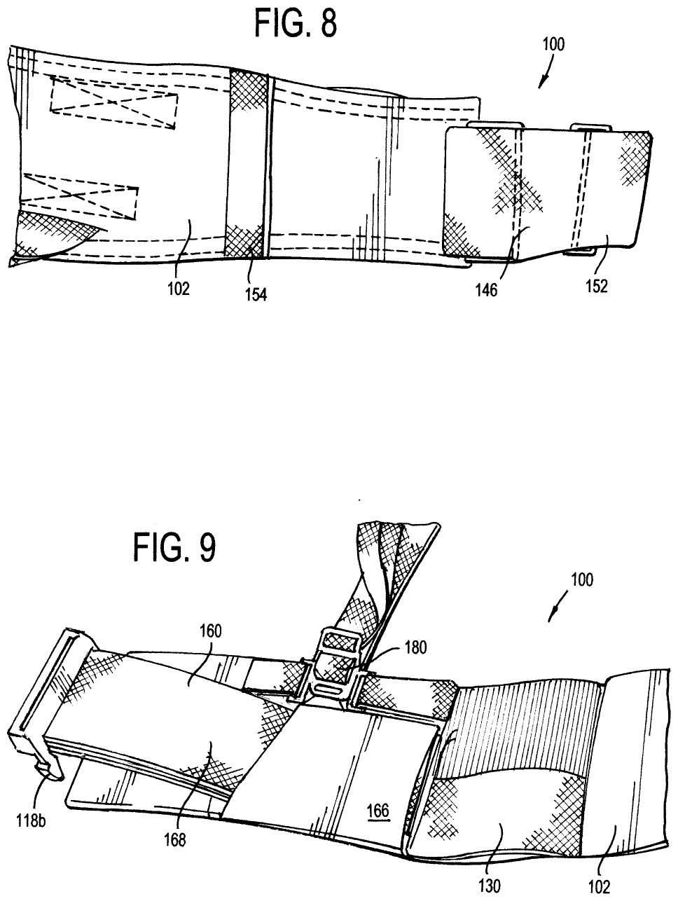

An adjustment feature 140 can be provided at one or both of the first and second ends 110 and 112 of the interface 100 that allows tightening or loosening of the sleeve 102 for sizing and fit adjustment for the user. FIGS. 6A and 7A illustrate the sleeve 102 of the interface 100 in a loosened state and FIGS. 6B and 7B illustrate the sleeve 102 in the tightened state. The sleeve 102′ of the interface 100′ can similarly be in a loosened state and a tightened state.

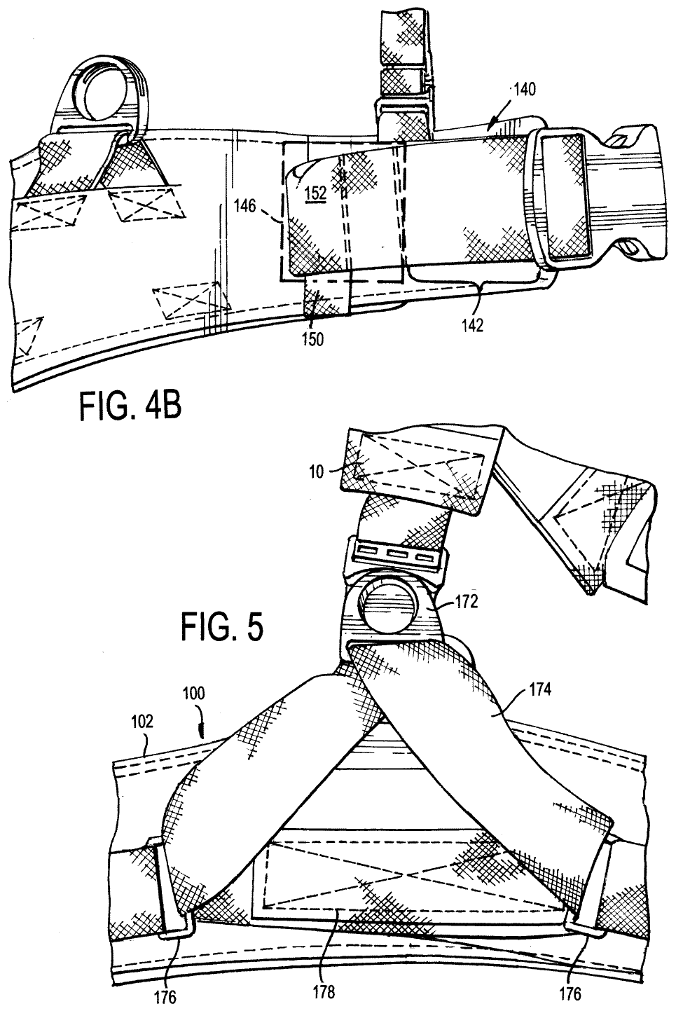

In an example, an adjustment feature 140 of interface 100 may be located at the first end 110 of the sleeve 102, as seen in FIGS. 4A and 4B, and may comprise a strap adjustment such as piece of webbing 142 (that can be 2 inches tall, for example) that is associated with female buckle component 118a, and a free end portion 146 of the webbing 142, which may be a pull tab for example (that can be 1.5 inches long, for example). The “pull tab” is also referred to in this disclosure as a strap used for tightening the sleeve. The webbing 142 is routed through a strap adjuster 148 (which is an end portion of the buckle component 118b) and terminates at the pull tab 146, as shown in FIGS. 4A and 4B. The strap adjuster 148 can be actuated (e.g., rotated and/or pulled) to cause the interface 100 to loosen, as the webbing 142 is pulled back through the strap adjuster 148. The strap adjuster 148 prevents the free end portion or pull tab 146 from passing through the buckle component 118b upon loosening, to prevent over-loosening. In an example, the adjustment feature 140 can provide a predetermined length of loosening 144, e.g. about 2 to 3 inches, around the body segment, such as the thigh. That allows the user to quickly and easily loosen the interface 100 or interfaces 100, e.g. thigh sleeves, a predetermined maximum amount, to improve thermal comfort and breathability, without adding undue snag risk. The strap adjuster 148 is designed to prevent the user from over-loosening the sleeve 102.

The pull tab 146 could be replaced with an alternative component that prevents the webbing 142 from displacing beyond a fixed amount. The strap adjuster 148 could be replaced with an alternative mechanism, such as a clasp, clip or clutch, which, when moved or triggered, allows the webbing 142 to displace so that the interface 100 can loosen.

The interface 100′ of the second embodiment can have an adjustment feature 140′ at one end of the sleeve 102′, as seen in FIGS. 10A and 11A. The adjustment feature 140′ may be an adjustable strap 142′ that can couple to one of the buckle components 118a or 118b. Having the adjustment feature 140′ at just one end provides simplicity in design and ease of use.

This loosening/tightening adjustment features 140 or 140′ would be used when switching from unassisted or disengaged mode of the wearable assistance device. In an example of the interface 100 or 100′ being used as a thigh sleeve, the user can loosen their thigh sleeves for bathroom breaks, to do paperwork in between lift tasks, lunch breaks, etc. This concept could be applied and provide benefit to any type of body interface of a wearable assistance device. This feature is especially useful for end-users that would need to wear the wearable assistance device for extended periods of time with periodic or frequent breaks in-between, for end-users that work in warm environments, or which have blood flow issues, and/or that have abrasive uniforms. The amount of loosening needed will vary based on multiple factors, including, for example, what part of the body the interface 100 or 100′ is intended for, the weight of the end-user, gender of the end-user, and other factors. The amount of loosening may be larger or smaller than the loosening range described in any of the examples or embodiments described herein.

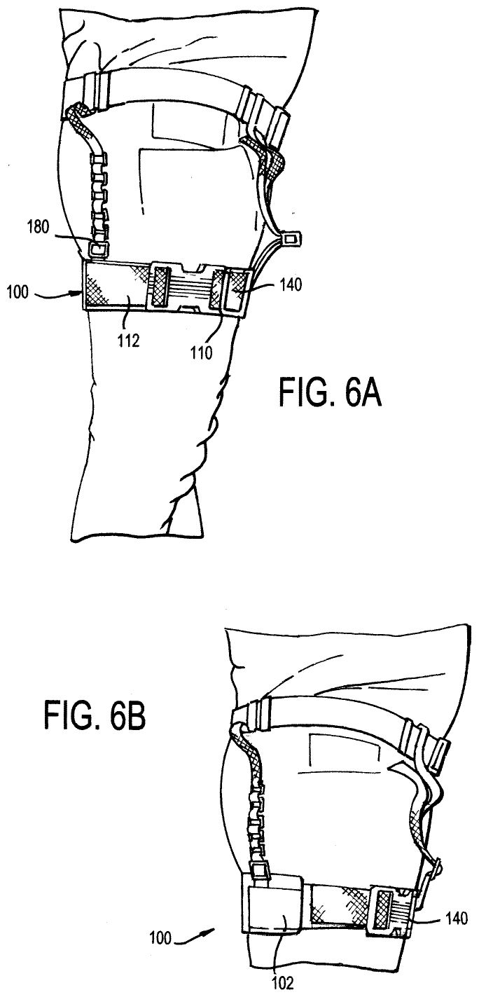

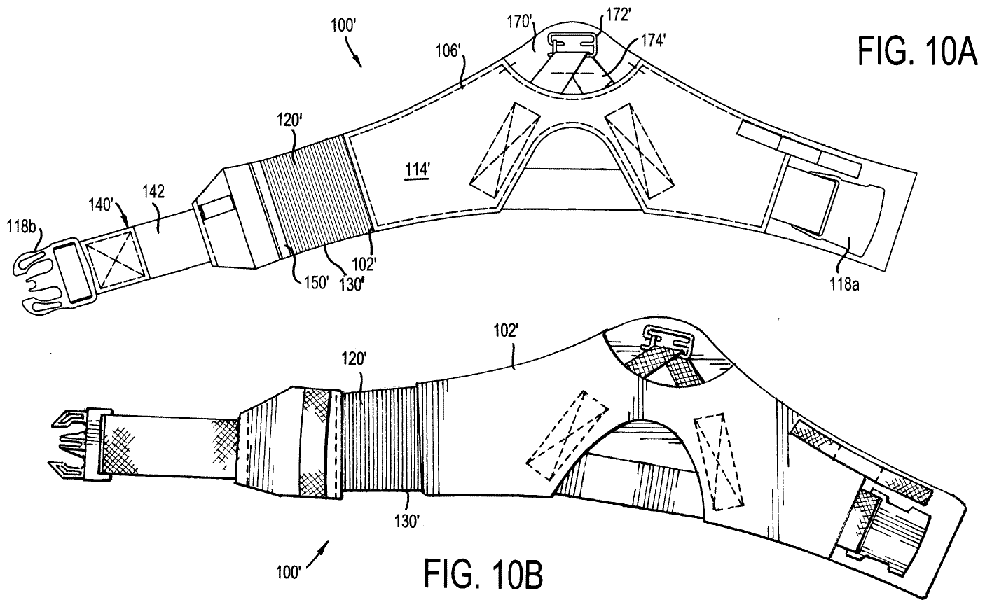

The adjustment feature 140 or 140′ can incorporate a strap management feature 150 or 150′. For example, as seen in FIGS. 5 and 8, the strap management feature 150 comprises one or more magnets 152 in or on the webbing free end portion or pull tab 146 and one or more small metal plates 154, such as steel plates, in or on the main body 104 of the sleeve 102 which engage the magnets via magnetic attraction, to retain the pull tab 148 on the sleeve 102. That magnet fastening (e.g., the combination of magnet(s) in/on the pull tab plus the steel plate in/on the sleeve 102) provides a way to consistently and/or easily tighten the sleeve 102 a predetermined amount, which is accomplished by pulling the pull tab 148 with the magnet 152 back to the point where the pull tab 146 magnetically attaches to the metal plate 154 positioned in or on the sleeve 102. That magnet engagement also provides strap management by holding the webbing 142 against the body, e.g. the leg, to prevent the webbing 142 from flapping around or becoming a snag risk.

When the user tightens the sleeve 102, the pull tab 146 (i.e., once pulled) automatically engages the steel plate 154, thereby preventing snag risks and dislodging of the webbing 142. If the pull tab 146 is knocked off the metal plate 154, the magnets 152 automatically return to the metal plate 154. The metal plate 154 can be made of steel (or other metal that is attractive to a magnet). When the steel plate 154 is exposed (when the sleeve 102 is loose), the steel plate 154 will not be attracted to anything metal in the environment. The quick loosening pull-tab 146 can be designed to have the magnets 152 facing the body, so that the pull tab 146 is less likely to be attracted to any metal in the surrounding environment.

The placement of the magnet 152 and steel plate 154 can be reversed such that the magnet 152 is in/on the main body 104 of the sleeve 102, and the steel plate 154 is on the webbing pull tab 148. Alternatively, the steel plate 154 can be replaced by a second magnet so that two opposing magnets are utilized. Other interlocking magnetic buckles or clips can also be used.

The strap management feature could also employ non-magnetic closures, such as side release buckles, zippers, snaps, buttons, hook-and-loop fasteners, reusable adhesives, hooks, and the like. For example, the strap management feature 150′ of the interface 100′ of the second embodiment can be a strap segment or loop 154′ that spans the height/width of the end of the sleeve 102 and is positioned to receive the pull tab 146′ of the adjustable webbing 142′, as seen in FIGS. 10A and 10C. The strap management feature 150′ provides simplicity and ease of manufacturing.

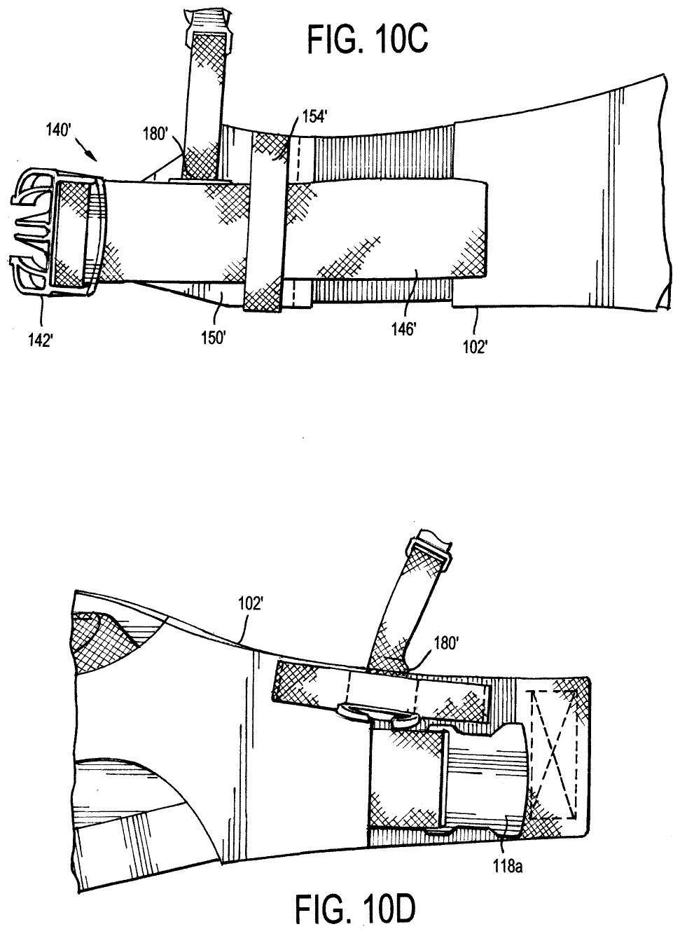

Another adjustment feature may be provided at the other end of the sleeve. For example, as seen in FIG. 9, another adjustment feature 160, such as an adjustable strap, may be provided on the other end, which is the second end 112 of the sleeve 102 of the interface 100 of the first embodiment, to provide further sizing and fit adjustability. That allows users to customize the sizing/fit to their own personal measurements (e.g., thigh size). Proper fit is important for the exosuit lower body interface to stay securely in place, and to fit and function when assistive force is applied. In an example, the strap 160 can be coupled to the male buckle component 118b. In an example, the strap 160 is webbing and the webbing may be about 2 inches in height/weight. The sleeve 102 can have a built-in pocket 166 that receives an end 168 of the strap. The built-in pocket 166 is configured for storing excess webbing, to mitigate snag risk or straps flapping. The pocket 166 can be made of a plain weave fabric or the like. The strap 160 could be beneficial for instances where the wearable assistance device may need to fit a variety of sizes (e.g., mass produced exosuits that come in limited sizes, several people may share an exosuit, end-user wears the exosuit with different types of clothing, etc.).

The function of fit adjustment for the interface 100 or 100′ can be achieved in many ways, including, but not limited to the adjustable strap on the buckle component 118b (as shown in FIG. 9), a hook and loop strap adjuster, Boa fit system, ratchet buckle, drawstring, and the like.

The interface 100 or 100′ may include one or more anchors 180 or 180′ as a way to maintain the interface's position on the body segment, such as to prevent the interface from slipping down the wearer's leg, for example, when the interface 100 or 100′ is loosened, such as for thermal comfort. The term “anchor” refers to the combination of webbing strap and fasteners (or functionally similar elements) that mechanically connect the interface 100 or 100′ (e.g., thigh sleeve) to another point on the user, their clothing or gear (e.g., their belt) to prevent slippage of the interface 100 or 100′ down the body segment due to, for example, gravity. The anchor strap may include elastic webbing or inelastic webbing, or both. In an example, the one or more anchors can be attached at or near the top 106 or 106′ of the sleeve 102. The anchors 180 or 180′ can be connectable to the user's belt, for example, such as via a strap that has a belt clip, magnet, snap, button, carabiner, side release clip, hook, buckle, or other like fastener. An example includes two anchors 180 or 180′ per interface 100 or 100′, as seen in FIGS. 3A and 6A and FIG. 10A. In another example, the anchors that can attach to a user's pant pockets via a pocket clip, magnet, snap, button, carabiner, side release clip, hook, buckle, or other like fastener. Alternatively, a custom belt can be designed for the wearable assistance device, with the anchors integrated therein that attach to the thigh sleeves.

Two or more anchors 180 or 180′ can be on each interface 100 or 100′, e.g. thigh sleeve. The anchors 180 or 180′ can be, for example, at least 90 degrees apart (and preferably 180 degrees apart) circumferentially about the thigh sleeve to prevent sagging or twisting of the thigh sleeve on the leg when it is in a loosened state. In an example, a first anchor 180a can be on the inner thigh and a second anchor can be on the outer thigh 180b, as seen in FIG. 3A.

There are also multiple possible examples of anchors that can be used with the interface embodiments of the present disclosure, including but not limited to elastic webbing with a hard stop made from a piece of inelastic webbing in parallel; elastic webbing with a soft stop made from a piece of elastic webbing in parallel; stiff webbing or cording; or bungee cording. These anchors can attach to the interface in multiple ways, including but not limited to suspender clips, side release buckles, magnets, snaps, buttons, hooks, pegs, hook and loop, and the like.

An engagement feature 170 or 170′ is provided on the sleeve 102 or 102′ for connecting the interface 100 or 100′ to a component 12 (FIG. 12A) of the wearable assistance device 10. The engagement features 170 and 170′ can be located in the middle portion of the sleeve. In an example, the engagement feature 170 may comprise, for example, a release buckle or snap mechanism 172, as seen in FIG. 5, or the engagement feature 170′ may be a clip or carabiner 172′, as seen in FIG. 10A and 11A. The engagement feature 170 or 170′ can be attached to the exterior side 114 or 114′ of the sleeve 102 or 102′ by webbing 174 or 174′, for example, as seen in FIG. 5 and FIG. 10A. The webbing 174 or 174′ can form an upside-down V and either be fixed to the sleeve 102 or 102′, such as via stitching, or can be free moving and coupled to the sleeve via guide loops 176 (FIG. 5). The release buckle 172 or clip 172′ is configured to engage a corresponding mechanism on a component 12, such as an elastic band or strap, of the assistance device 10, as shown in FIG. 3A.

When the interface is worn by the user as a thigh sleeve, the upside-down V-shaped webbing 174 or 174′ will be positioned on the back of the leg with the elastic band of the assist device and the buckle or carabiner 172 or 172′ running down to the rest of the thigh sleeve. This upside-down V provides the load path that helps ensure the interface stays secured fixed to the thigh of the user without sliding up the leg when force is applied by the wearable assist device. In some embodiments, when the force in the elastic band of the assist device increases, this tightens the sleeve onto the leg (because the free moving webbing 174 or 174′ can move through the webbing guides 176). A stitched-down webbing 178 (FIG. 5) in the middle of the upside-down V-shaped webbing 174 or 174′ can prevent over-tightening of the thigh sleeve. The angle of the V-shape, which may be between about 30 to 120 degrees, achieves the desired load path and performance of the interface under load.

In an example, for additional comfort, ventilation can be added to the interface 100 or 100′. For example, the sleeve 102′ of the interface 100′ can include a ventilated panel 103′, as seen in FIGS. 11A and 11C for improved thermal comfort when worn by the user. In another example, the interior side 116′ of the sleeve 102′ of the interface 100′ can have a rubberized non-slip fabric across a portion or the entire side thereof to reduce twisting on the body segment, e.g. leg, and improve comfort and force transfer.

In an embodiment, it is possible for the interface 100 to be directly integrated into clothing through both permanent and temporary configurations. For example, channels or pockets can be created in clothing in which the interface 100 could be inserted. Examples of permanent configurations include but are not limited to the lower body interface sewn into pants, shorts, undergarments, or other clothing or gear. Examples of temporary configurations include but are not limited to: hook and loop, zippers, hooks, buttons, snaps, magnets, and the like that connect the thigh sleeve to the pants. Also, the embodiments of the present disclosure can be with or without a sizing/fit adjustment feature.

In another embodiment, the sleeve can include only one or more of the elastic portions in the interface, without a separate stop.

Additional embodiments can also include a clutch mechanism or powered actuator (e.g., motor) being directly integrated into the interface 100. The clutch could be powered or un-powered. The loosening and/or tightening of the interface 100 could actuate separately from the clutch, or could actuate simultaneously with the clutch, such that when the assistance device is disengaged or not assisting, the body interface 100 is loosened, and/or when the assistance device is engaged or is assisting, the interface 100 is tightened. Separate actuation of the clutch or powered actuator involves affixing the clutch to the interface 100 (e.g., thigh sleeve). Simultaneous actuation could be achieved with a passive or powered (e.g., motorized) mechanism. For instance, when the webbing pull tab 146 is pulled to tighten the interface 100 (FIG. 7A), that pull-tab 146 could also be attached to a separate cable that runs to the clutch to pull it into engagement (e.g., pulling a pawl into a rachet, or pulling the cam of a friction clutch into contact). A bias or reset spring in the clutch would then cause the clutch to disengage when the interface 100 was loosened (FIG. 7B), for instance, due to slackening of the cable that physically pulls the clutch into engagement. Alternatively, one or more motors could pull the webbing pull tab 146 to tighten the interface 100 and at the same time pull the clutch into engagement, or trigger another power actuator to provide assistance.

The method steps in any of the embodiments described herein are not restricted to being performed in any particular order. Also, structures or systems mentioned in any of the method embodiments may utilize structures or systems mentioned in any of the device/system embodiments. Such structures or systems may be described in detail with respect to the device/system embodiments only but are applicable to any of the method embodiments.

Features in any of the embodiments described in this disclosure may be employed in combination with features in other embodiments described herein, such combinations are considered to be within the spirit and scope of the present disclosure. The contemplated modifications and variations specifically mentioned in this disclosure are considered to be within the spirit and scope of the present disclosure.

More generally, even though the present disclosure and exemplary embodiments are described above with reference to the examples according to the accompanying drawings, it is to be understood that they are not restricted thereto. Rather, it is apparent to those skilled in the art that the disclosed embodiments can be modified in many ways without departing from the scope of the disclosure herein. Moreover, the terms and descriptions used herein are set forth by way of illustration only and are not meant as limitations. Those skilled in the art will recognize that many variations are possible within the spirit and scope of the disclosure as defined in the following claims, and their equivalents, in which all terms are to be understood in their broadest possible sense unless otherwise indicated.

As used in this specification and the appended claims, the singular forms “a”, “an” and “the” include plural referents, unless the context clearly dictates otherwise. Similarly, the adjective “another,” when used to introduce an element, is intended to mean one or more elements. The terms “comprising,” “including,” “having” and similar terms are intended to be inclusive such that there may be additional elements other than the listed elements.

Additionally, where a method described above or a method claim below does not explicitly require an order to be followed by its steps or an order is otherwise not required based on the description or claim language, it is not intended that any particular order be inferred. Likewise, where a method claim below does not explicitly recite a step mentioned in the description above, it should not be assumed that the step is required by the claim.

It is noted that the description and claims may use geometric or relational terms, such as right, left, above, below, upper, lower, top, bottom, linear, arcuate, elongated, parallel, perpendicular, etc. These terms are not intended to limit the disclosure and, in general, are used for convenience to facilitate the description based on the examples shown in the figures. In addition, the geometric or relational terms may not be exact. For instance, walls may not be exactly perpendicular or parallel to one another because of, for example, roughness of surfaces, tolerances allowed in manufacturing, etc., but may still be considered to be perpendicular or parallel.

What is claimed is:

1. An interface for a wearable assistance device, comprising:
a sleeve comprising,
a main body configured to at least partially surround a user's body segment, the main body having at least one elastic portion,
a stop positioned adjacent to the at least one elastic portion such that the stop bridges the at least one elastic portion, wherein the stop is configured to restrict an amount of stretch that the elastic portion affords the sleeve beyond a set length, and
an engagement feature configured to engage a component of the wearable assistance device.

2. The interface of claim 1, wherein the stop is an inelastic webbing.

3. The interface of claim 1, wherein the stop is an elastic material having a stiffness greater than a stiffness of the at least one elastic portion.

4. The interface of claim 1, wherein a length of the stop is longer than a length of the at least one elastic portion such that the stop has slack when not in use.

5. The interface of claim 1, wherein the main body includes a top, a bottom, and opposite ends, and a length defined between the opposite ends, the length being longer than a height of the sleeve defined between the top and bottom.

6. The interface of claim 5, wherein the stop is located at the bottom of the main body.

7. The interface of claim 5, wherein the opposite ends are configured to connect to one another.

8. The interface of claim 5, wherein a middle portion of the main body is wider than the first and second ends, and the engagement feature is located at the middle portion and extends away from the bottom of the main body.

9. The interface of claim 8, wherein the engagement feature is a release buckle or a carabiner attached to the sleeve.

10. The interface of claim 5, wherein the interior side of the main body is formed of non-slip fabric.

11. The interface of claim 1, wherein the main body includes at least one ventilated panel.

12. The interface of claim 1, wherein the sleeve is a thigh sleeve.

13. A wearable assistance device comprising the interface of claim 1.

14. An interface for a wearable assistance device, comprising
a sleeve comprising:
a main body configured to at least partially surround a user's body segment, the main body having at least one elastic portion,
at least one adjustment feature coupled to the main body, the adjustment feature comprising a webbing or strap that has a free end portion thereof,
a strap management feature positioned on the main body, the strap management feature being configured to removably secure the free end portion to the sleeve, and
an engagement feature configured to engage a component of the wearable assistance device,
wherein the free end portion is configured to tighten the sleeve on the user's body segment and to be secured via the strap management feature, and
wherein the adjustment feature is configured to limit an amount of loosening of the sleeve.

15. The interface of claim 14, wherein the strap or webbing runs through a strap adjuster wherein actuation of the strap adjuster removes at least a portion of the free end portion from the strap management feature to thereby loosen the sleeve on the user's body segment and the loosening of the sleeve is limited by the free end portion on the strap contacting the strap adjuster.

16. The interface of claim 14, further comprising a stop positioned adjacent to the at least one elastic portion such that the stop bridges the at least one elastic portion, wherein the stop is configured to restrict an amount of stretch that the elastic portion affords the sleeve beyond a set length.

17. The interface of claim 14, wherein the strap management feature is configured to removably secure the free end portion to an exterior side of the main body.

18. The interface of claim 17, wherein the strap management feature is a strap segment spanning a width of an end of the main body.

19. The interface of claim 17, wherein the strap management feature removable secures the free end portion via magnetic attraction between a magnet positioned in or on the free end portion and a metal plate in or on the main body, or vice versa.

20. The interface of claim 19, wherein the metal plate is a first metal plate, wherein the interface further comprises a second metal plate also positioned in or on the main body at a location that is different than the position of the first metal plate in or on the main body, wherein the free end portion is configured to be removably secured to the main body via magnetic attraction between the magnet and the first metal plate, when tightening the sleeve around the body segment, and wherein the free end portion is configured to be removably secured to the main body via magnetic attraction between the magnet and the second metal plate, when loosening the sleeve around the body segment.

21. The interface of claim 14, further comprising one or more anchors configured to connect or suspend the sleeve from the user's clothing, belt, or other body-worn gear.

22. The interface of claim 21, wherein one of the anchors is positioned along an interior side of the sleeve and another one of the anchors is positioned along an exterior side of the sleeve.

23. The interface of claim 14, wherein the strap or webbing of the adjustment feature is provided at one end of the sleeve.

24. The interface of claim 23, wherein an adjustable strap is provided at the other end of the sleeve.

25. The interface of claim 14, wherein the sleeve is a thigh sleeve.

26. A wearable assistance device comprising the interface of claim 14.
